(19) United States
(12) Patent Application Publication
(10) Pub. No.: US 2010/0076346 A1
(43) Pub. Date: Mar. 25, 2010

(54) Method and System for Energy Returning Ankle Foot Orthosis (ERAFO)

(75) Inventors: Mark F. Abel, Earlysville, VA (US); Paul E. Allaire, Charlottesville, VA (US); Bradford C. Bennett, Charlottesville, VA (US); Adam C. Wolovick, Richmond, VA (US)

Correspondence Address: UNIVERSITY OF VIRGINIA PATENT FOUNDATION, 250 WEST MAIN STREET, SUITE 300, CHARLOTTESVILLE, VA, 22902, US

(73) Assignee: UNIVERSITY OF VIRGINIA PATENT FOUNDATION, Charlottesville, VA (US)

(21) Appl. No.: 11/993,583

(22) PCT Filed: Jun. 29, 2006
(86) PCT No.: PCT/US06/25515

§ 371 Date: Dec. 21, 2007

Related U.S. Application Data
(60) Provisional application No. 60/695,346, filed on Jun. 30, 2005.

(51) Int. Cl. A61B 5/103; A61H 1/02

(52) U.S. Cl. 600/592; 601/27

(57) ABSTRACT

An orthotic device/system and method including at least one energy storage component and at least one trigger component which prevents release of the stored energy until the desired point in the movement cycle of an extremity. Use of the device/system and method permits, among other things, the orthosis to capture energy from the force present between a patient and a supporting surface and return it at a specified point in the movement cycle of the subject's extremity.

### CROSS-REFERENCE TO RELATED APPLICATION

This application claims the benefit of priority of U.S. Provisional Application Ser. No. 60/695,346, filed on Jun. 30, 2005, entitled “Method And System For Energy Returning Ankle Foot Orthosis (ERAFO),” of which is hereby incorporated by reference herein in its entirety.

### FIELD OF THE INVENTION

The present invention relates generally to orthotic devices, and, more particularly, to orthotic devices which capture and return mechanical energy to assist the subject with certain motions.

### BACKGROUND OF THE INVENTION

Orthotic devices are typically prescribed to serve as supports or braces for weak or ineffective joints or muscles. Some conventional orthoses allow limited motion of one or more of a subject's joints, often in one plane, by connecting the rigid portions of the orthosis with hinges or incorporating a flexible member which is capable of being bent as the subject moves.

More advanced conventional orthoses utilize various elastic materials to capture and return mechanical energy as the subject moves the device. For example, U.S. Patent Application Publication No. US 2004/0186401 A1 (of which is hereby incorporated by reference herein in its entirety) allegedly discloses one such passive orthotic device incorporating a leaf spring as an energy storage mechanism. U.S. Patent Application Publication No. US 2005/0054959 A1 (of which is hereby incorporated by reference herein in its entirety) allegedly discloses a similar passive orthotic device, also using a spring mechanism. U.S. Pat. No. 6,228,043 (of which is hereby incorporated by reference herein in its entirety) allegedly discloses another passive ankle orthosis device which is built into an athletic shoe. U.S. Pat. No. 5,475,935 (of which is hereby incorporated by reference herein in its entirety) allegedly discloses a “jumping assist system” that captures energy as the wearer moves into the “jump ready” position and then uses the energy to assist the wearer in jumping. U.S. Patent Application Publication No. US 2005/0059908 A1 (of which is hereby incorporated by reference herein in its entirety) allegedly discloses an apparatus for assisting in body movement which includes an exoskeleton framework and an elastic force transmission system. Although some prior art orthotic systems may possibly have sought to capture energy during one portion of the gait cycle and return it at another point in the cycle, these devices deliver suboptimal assistance to many subjects because they are passive in that they do not return the captured energy at the most advantageous point in the gait cycle.

Other conventional orthotic devices may possibly attempt to control the movement of one or more joints by using various locking and braking systems. U.S. Pat. No. 5,121,742 (of which is hereby incorporated by reference herein in its entirety) allegedly discloses a “lower extremity orthotic device”, one embodiment of which includes a locking knee joint. U.S. Pat. No. 4,632,096 (of which is hereby incorporated by reference herein in its entirety) allegedly discloses an orthotic device which unlocks the knee joint when the ankle joint reaches certain angles. While these devices may possibly aid the subject with certain motions to and restrict undesired movements, they do not take advantage of potentially excess mechanical energy which can be used to aid the subject's movements.

Further advanced conventional orthotic devices attempt to combine energy storage with locking joints. See, for example, Gharooni, Heller, and Tokhi, A New Hybrid Spring Break Orthosis for Controlling Hip and Knee Flexion in the Swing Phase, IEEE Transactions on Neural Systems and Rehabilitation Engineering, Vol. 9, No. 1, March 2001. (of which is hereby incorporated by reference herein in its entirety) The article allegedly discloses an orthotic device that utilizes excess torque produced by the quadriceps muscles to, charge a torsion spring during the gait cycle. A braking device holds the spring in its deflected position, storing mechanical energy, until later in the gait cycle when the spring is released and the resulting force aids the wearer in flexing his or her knee so as to produce adequate foot-ground clearance. As discussed in the article, the utility of this design is limited by its size and complexity. Additionally, the device requires the subject to provide the stored energy from muscular effort. Finally, this device does not deliver the energy in a manner that aids the subject in propelling herself—it merely helps her develop adequate foot-ground clearance.

In the specific case of ankle foot orthoses (AFOs), many prior art devices only provide subjects with small improvements in walking capability. Most of the improvements from the use of conventional AFOs may be a result of the brace holding the ankle in a position which to moves the point of foot contact towards the heel. Many individuals presently using conventional AFOs would be aided in their walking by additional ankle torque at push-off.

Conventional AFOs are typically passive braces constructed of rigid materials, such as polypropylene, to resist abnormal postures and maintain optimum position. Research has shown that most polypropylene AFOs behave as linear springs in both dorsi- and plantarflexion. Thus a stiffer brace restricts ankle motion and the ability of the ankle to apply walking force, while a more flexible brace may not provide sufficient support or be able to store sufficient energy to improve walking. Prior art AFOs are suboptimal because they do not act to both capture energy during limb loading and return a portion of that energy at the time of push-off.

In typical walking energy is initially dissipated on foot contact by the heel pad and within the shoe while the muscles of the anterior shank dissipate load energy as the foot moves from heel strike to foot flat. Then the calf muscles dissipate or work eccentrically in mid-stance until initiation of push off when they concentrically contract and return any elastic energy to provide propulsion. At low and moderate walking velocities the dissipated power is greater than the power generated at push off creating the opportunity for the development of an energy returning AFO to improve walking energy costs.

In able-bodied walkers the plantar flexor moment at the ankle increases in a nearly monotonic fashion until it peaks at approximately 80% of stance phase. The situation with individuals with cerebral palsy (CP) is dramatically different. There is typically an external plantar flexor moment because initial floor contact is forward of the ankle (midfoot or forefoot) which is followed by an early peak in ankle moment near 25% of stance phase resulting in wasted energy. Additionally, the second peak of the ankle moment and propulsion in late stance is commonly reduced, thus contributing to reduced swing phase knee and foot flexion as well as reducing the horizontal trajectory of the body.

Conventional AFOs are typically prescribed to ameliorate these difficulties in walking for individuals with CP. However, conventional brace properties are largely generic and match the abilities of individuals in only the most general way and do not adjust to differing locomotion tasks. The conventional AFOs, by maintaining the ankle at the prescribed 90°, often reduce the early ankle moment by increasing ankle stiffness but also reduce power generation in the more vigorous ambulators (equinus group). Patients with slow crouched gait and poor baseline propulsion seem to benefit by increasing stiffness but no power is provide by the brace. The result is that the ankle moments are little changed by the currently prescribed AFOs.

None of the patents and patent applications described above provides the important advantages of capturing energy present due to the forces between the subject and a supporting surface and returning the energy to aid in locomotion.

### SUMMARY OF THE INVENTION

The present invention relates to an orthotic device which stores mechanical energy during one portion of a movement of an extremity and releases the stored mechanical energy at a predetermined point in another portion of the movement and, more particularly, to an ankle foot orthosis (AFO) which captures and stores mechanical energy in the early portion of the gait cycle and returns it later in the gait cycle to assist in toe-off or push-off.

Some of the various embodiments of the present invention brace or MO capture energy in the early portion of the gait cycle and return it later at the moment of toe-off or push-off. In addition, the various embodiments of the present invention AFO resist abnormal ankle-leg postures that are commonly seem in some neuromuscular conditions such as CP. Specifically, ankle extension or equinus ankle posture that is seen in early stance is resisted until the appropriate time in the gait cycle. The unique mechanical elements assembled to accomplish these objectives may include, for example but not limited thereto, an energy storage component and a trigger component.

In one embodiment, two cantilever springs (e.g., spring mechanism or any energy storage device/system or the like) beneath the foot that are linked through cables and controlled by one or more ratchets. As the rear cantilever spring is depressed to store energy, the front spring is elevated until forefoot pressure depresses it, later in the gait cycle, to trigger the release of the energy stored in the rear spring. Or said differently, the rear cantilever is depressed with foot contact to store energy until the elevated front beam depresses as the body weight transitions forward during gait thus releasing the energy stored in the rear beam. While this brace is helpful to augment the weak power found in patients with CP, the mechanics of the brace are used to enhance walking performance in other medical conditions and potentially even in persons without specific disabilities.

The various embodiments of the present invention active brace that is provided herein are intended for use by several subject populations. Initially, the primary group for whom we expect, the maximum benefit to be realized is children afflicted with CP. In addition, adult individuals who suffer from CP will be able to utilize this device and method. Furthermore, the various embodiments of the present invention mechanical device and method may be utilized in the development of prosthetic devices for amputees and for the improvement or enhancement in the walking efficiency of non-disabled individuals.

It is a first aspect of the present invention to provide an orthotic device including an energy storage component which stores mechanical energy generated by the forces between a subject and a supporting surface and a trigger component which prevents the release of the stored mechanical energy until a specific point in the movement cycle of an extremity of the subject.

In a more detailed embodiment of the first aspect, the device operates during the gait cycle of a subject. It should be appreciated that the gait cycle may include any particular intensity, style or speed of travel/movement of the subject, including running, jogging, walking or any combination thereof, variations in the range thereof, and any type of gait/movement utilized by the subject; and at various locations of the subject. In another more detailed embodiment of the first aspect, the released energy assists the subject in locomotion. In yet another more detailed embodiment of the first aspect, the device is an ankle-foot orthosis (AFO). It should be appreciated that the AFO may be applied to limbs or limb areas (and associated joints) including expanded up the leg or used in a prosthetic foot or leg, for example. It should be appreciated that the device and related method discussed herein may be applied to any limbs or portions thereof including arms. In another more detailed embodiment of the first aspect, the released energy assists the subject with plantar flexion. In to still another more detailed embodiment of the first aspect, the release of stored mechanical energy occurs when the body weight of the subject is partially or fully over the forefoot. In another more detailed embodiment of the first aspect, the energy storage component is comprised of at least one spring. In a further detailed embodiment, the energy storage component spring is a cantilever spring.

In another more detailed embodiment of the first aspect, the trigger component is actuated by a spring reaching a predetermined deflection. In a further detailed embodiment, the trigger component spring is a cantilever spring.

It is a second aspect of the present invention to provide a method of using an orthotic device which is comprised of an energy storage component which stores mechanical energy generated by the forces between a subject and a supporting surface and a trigger component which prevents the release of the stored mechanical energy until a specific point in the movement cycle of an extremity of the subject.

Further, an aspect of the various embodiments of the present invention a method of using a orthotic device disposed on a subject. The method comprising: storing mechanical energy generated by the forces between the subject and a supporting surface during a portion of a movement cycle; and preventing the release of the stored mechanical energy until a specific point in the movement cycle. In an approach, the storage of mechanical energy is provided by an energy storage component. In an approach, the release prevention is provided by a triggering component.

The invention itself, together with further objects and attendant advantages, will best be understood by reference to the following detailed description taken in conjunction with the accompanying drawings.

### BRIEF DESCRIPTION OF THE DRAWINGS

The foregoing and other objects, features, and advantages of the present invention, as well as the invention itself, will be more fully understood from the following description of preferred embodiments, when read together with the accompanying drawings, in which:

FIG. 1 is a block diagram showing the components of the present invention and their interactions generally.

FIG. 2 is a schematic diagram depicting the general arrangement of the various components of an exemplary embodiment of the present invention as applied to an ankle foot orthosis (AFO).

FIG. 3 shows a detailed schematic view of an exemplary embodiment of the present invention as applied to an AFO.

FIG. 4 is a detailed schematic view of an exemplary embodiment of the present invention as applied to an AFO, specifically showing the cable connection to the energy storage component.

FIG. 5 is a detailed schematic view of an exemplary embodiment of the present invention as applied to an AFO, specifically showing the cable connection to the trigger component.

FIG. 6 is a detailed schematic view of an exemplary embodiment of the present invention as applied to an AFO, specifically showing the upper portion of the AFO and the cable connections.

### DETAILED DESCRIPTION OF THE INVENTION

The exemplary embodiments described herein relate to orthotic devices and methods, for example and without limitation, to an ankle foot orthosis (AFO). It will be apparent to those of ordinary skill in the art that the devices and processes disclosed herein may be useful for other types of orthotic devices which serve as supports or braces for weak or ineffective joints or muscles, or which assist amputees or non-impaired subjects with one or more movements.

In order to more clearly and concisely describe the subject matter of the claims, the following definitions are intended to provide guidance as to the meaning of specific terms used in the following written description, examples, and appended claims.

As used herein, the term “extremity” includes a limb of the body, particularly a human hand or foot; or any body part/location as applied in desired or required operation of the present invention device and method.

As used herein, the term “locomotion” means the act of moving from place to place.

As used herein, the term “supporting surface” includes the ground, a floor, a step, a stool, a chair, a table, or any other surface to which a subject may apply a force to resist motion due to the force of gravity.

As used herein, the term “orthotic device” includes any device used as a support or brace for joints or muscles.

As used herein, the term “subject” includes any person or animal on which an orthotic device may be fitted.

As used herein, the term “gait cycle” means the rhythmic alternating movements of the 2 lower extremities (or other body extremities), which result in the forward or other movement of the body; for example, the manner in which we walk, jog or run; or any combinations/variations thereof.

As used herein, the term “cantilever spring” means a spring that is comprised of a member that projects beyond a fulcrum and which is supported by a force behind the fulcrum.

As used herein, the term “free end” means the unsupported end of a cantilever spring which is deflected by forces applied to the spring.

As used herein, the term “fixed end” means the end of the cantilever spring which is supported in a fixed position behind the fulcrum.

As used herein, the term “movement cycle” includes any movement of an extremity which is conducted in a repetitive manner.

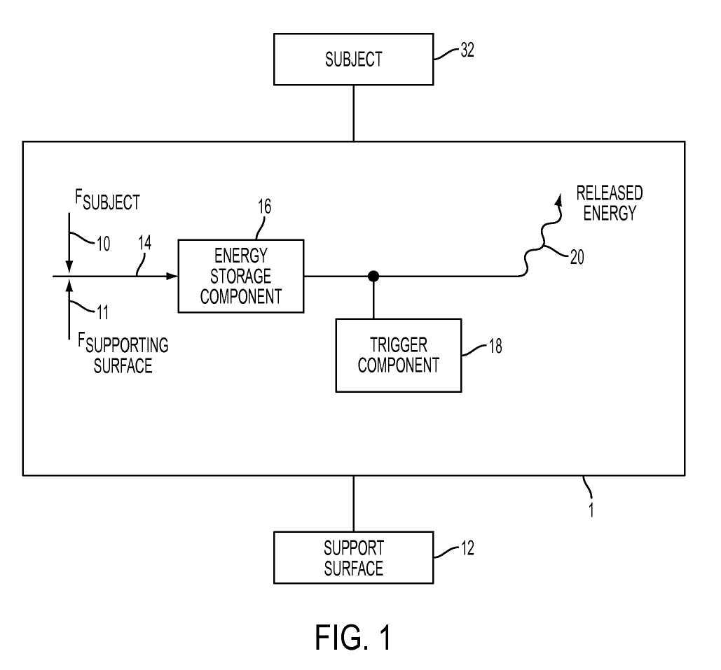

FIG. 1 provides a schematic illustration of an embodiment of the present invention orthotic system. Turning to FIG. 1, the forces 10, 11 present between a subject 32 and a supporting surface 12 (e.g., floor, chair, rail, step, etc.) provide mechanical energy 14 which is captured and stored in the energy storage component 16. The trigger component 18 prevents release of the stored mechanical energy until a predetermined point in a movement cycle, at which time the trigger component 18 is actuated and the stored energy is released. The released energy 20 is directed in such a manner as to assist the subject with a movement of an extremity.

For instance, in one approach, the present invention is applied to an AFO. The forces present between the subject and the support surface, possibly a floor, are captured in a cantilever spring located beneath the subject's heel (not shown). Another cantilever spring located beneath the subject's forefoot (not shown) acts as the trigger mechanism. As such, the energy is captured as the subject's heel strikes the ground and the spring under the heel is deflected. As the gait cycle continues, the subject's center of mass moves forward and the subject begins to load the spring under the forefoot. Deflection of the spring under the subject's forefoot actuates the trigger mechanism which releases the energy stored in the spring under the subject's heel. The released energy is transmitted via a cable to the ankle joint. The tension in the cable results in a torque at the ankle joint which assists the subject with plantar flexion at the point of push-off in the gait cycle. Once the subject's foot is lifted above the floor, the springs return to their initial positions and the cycle repeats.

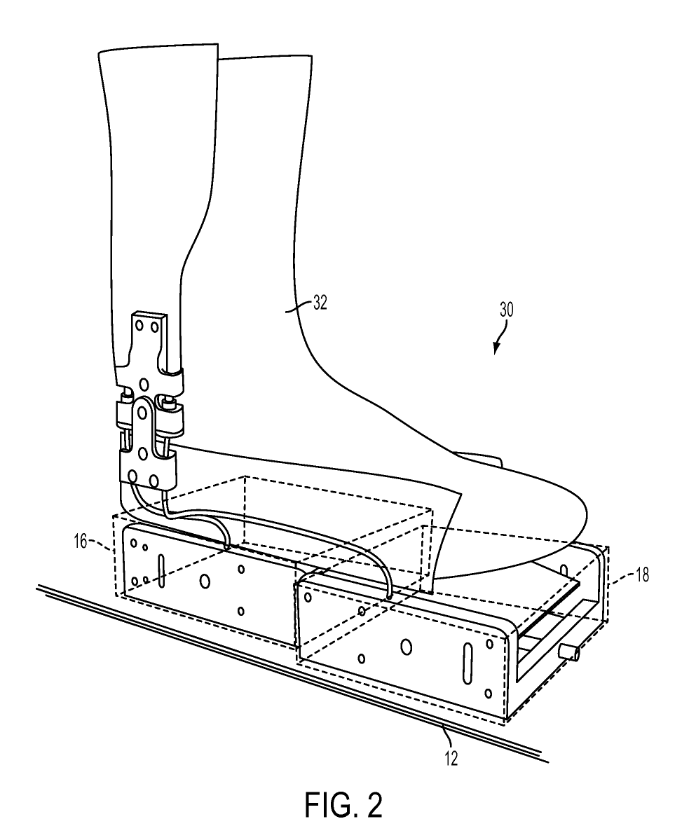

FIG. 2 shows an exemplary embodiment of the present invention as applied to an AFO. The subject's 32, for example, lower extremity is placed in the AFO 30 in the manner typical of conventional AFOs. In this exemplary embodiment, the energy storage component 16 is located between the subject's heel and the supporting surface 12. The trigger component 18 is located between the subject's forefoot and the supporting surface 12.

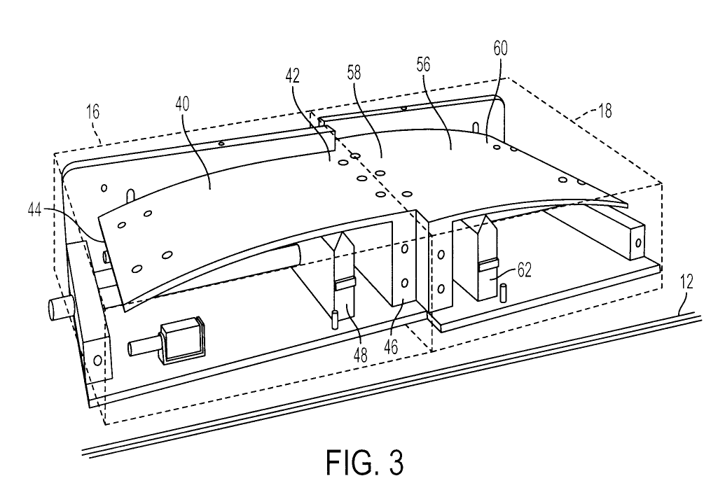

Turning to FIG. 3, the energy storage component 16 is comprised of a cantilever spring 40 which has a fixed end 42 and a free end 44. The fixed end 42 is rigidly connected to the frame of the AFO 46. The position of a fulcrum 48 is variable to allow adjustment for individual subjects. Similar to the energy storage component 16, the trigger component 18 is comprised of a cantilever spring 56 which has a fixed end 58 and a free end 60. A fulcrum 62 is adjustable to allow adaptation for individual subjects. It should be appreciated that other spring type mechanisms, resilient means, or energy storage device/systems or the like may be utilized in place of or along with the cantilever spring.

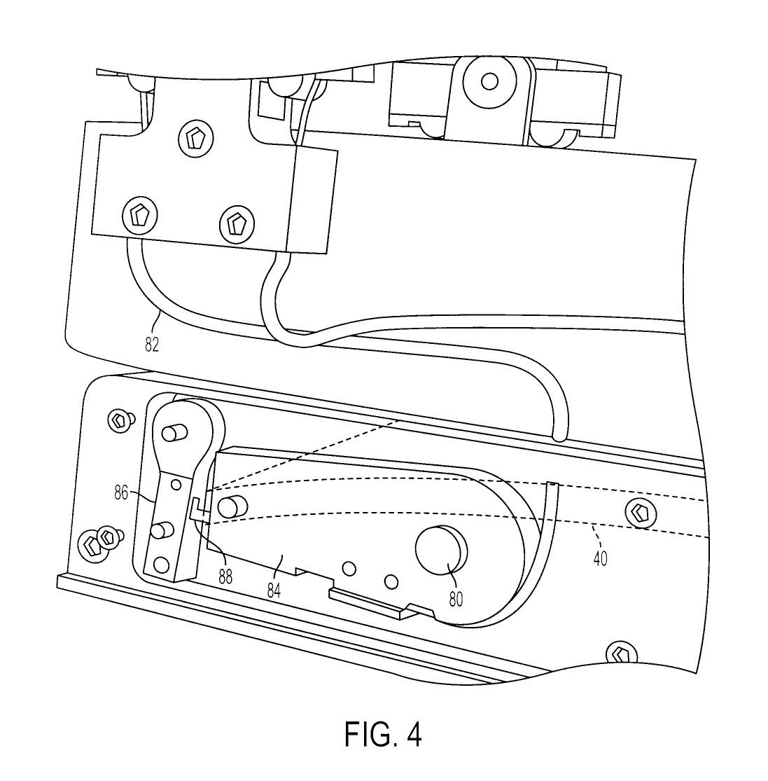

As shown in FIG. 4, the free end of the cantilever spring in the energy storage component is attached to a pivot 84 which rotates about an axis 80. As the pivot rotates, the tension on the cable 82 varies: as the free end of the cantilever spring is deflected, the tension on the cable decreases. Conversely, as the free end of the cantilever spring is allowed to return to its undeflected position, the tension on the cable increases. The pivots attached to the energy storage component cantilever spring 40 are designed with ratchet teeth 88 that mesh with a pawl element 86. The pawl element is linked via a cable to a small rocker arm located beneath the forward-facing beam. A similar pivot mechanism, constructed in a similar manner, is used on the opposite side of the AFO.

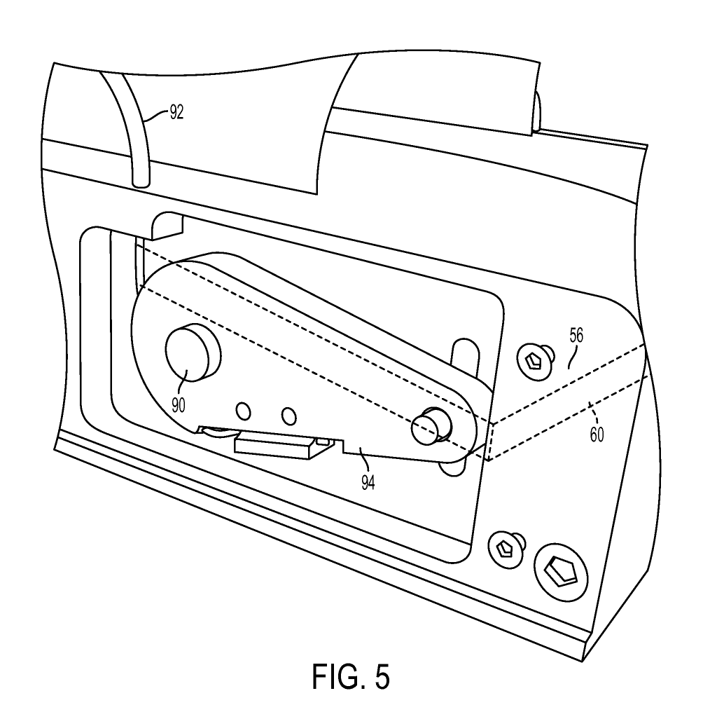

As shown in FIG. 5, the free end of the cantilever spring in the trigger component is attached to a pivot 94 which rotates about an axis 90. As the pivot 94 rotates, the tension on cable 92 varies: as the free end 56 of the cantilever spring 60 is deflected, the tension on the cable 92 decreases. Conversely, as the free end 56 of the cantilever spring 60 is allowed to return to its undeflected position, the tension on the cable 92 increases. The trigger component pivots 94 do not have a ratchet and pawl mechanism. A similar pivot mechanism, constructed in a similar manner, is used on the opposite side of the AFO.

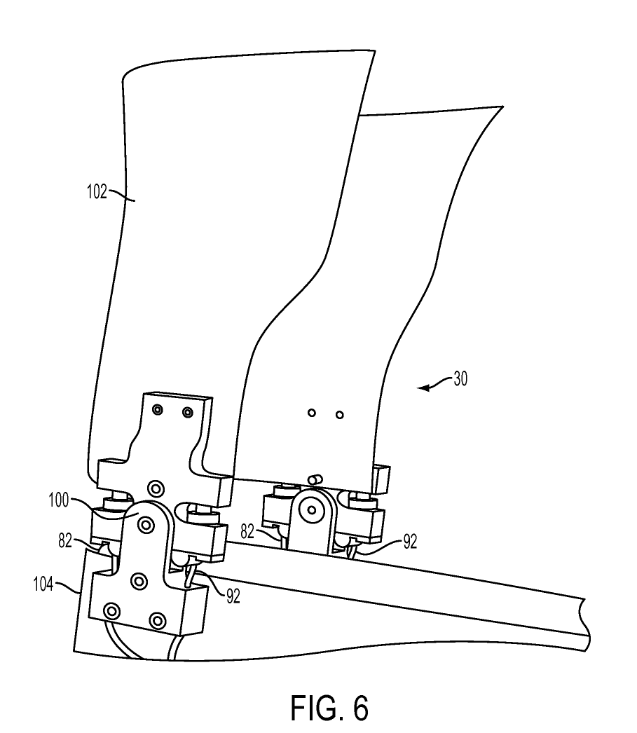

As depicted in FIG. 6, the cable 82 attached to the energy storage component and the cable 92 from the trigger component are fixed to the rear and front, respectively, of the upper portion of the AFO 30 above the ankle joint 100. The two major components of the ankle joint are the upper ankle joint piece 102 and the lower ankle joint piece 104. These elements are hinged together so that the upper piece 102 freely rotates in relation to the lower piece 104. The lower joint piece 104 is imbedded in the plastic section of the lower foot plate portion of the AFO in which the under-foot mechanical components are also located. This provides a rigid connection between the lower ankle joint piece and the under-foot mechanical components. The cables 82 & 92 are attached to the upper joint piece on the respective sides described above. The upper ankle joint piece is rigidly mounted to a polypropylene plastic upper section that is the norm on current hinged AFOs.

The present invention AFO device and method is provided to address the issues and constraints identified by previous research to reduce effort and metabolic cost of walking. First, it captures energy normally dissipated during the foot contact and initial stance phase and then releases it at the optimal time when the subject's plantar flexor torque will accelerate the person in the walking direction (forward). Thus, much as in running, as the body lowers during foot contact energy is stored and held until late in stance when it is returned to the ankle joint to increase the applied plantar flexor moment. Second, this AFO will allow for unrestricted dorsi-flexion through mid-stance to allow pendular movement of the center of mass (CoM) and transition of the CoM to the forefoot where push-off will promote a forward trajectory. Third, it provides dorsi-flexion assist during swing phase to minimize the spastic response of the triceps surae associated with equine gait foot contact. Fourth, it is lightweight, so as not to impede a subject's gait through excess mass. Finally, the applied torque, range of motion, and torque application timing are adjustable to adapt to the gait characteristics of each individual subject.

Research into the effectiveness of current AFO designs on the improvement in the gait patterns of children with CP indicates that the improvement in gait parameters is minimal. However, existing AFO's do provide an important benefit to this population. That is, they minimize the equinus gait pattern by controlling the amount of plantarflexion at the ankle joint, either rigidly or through mechanical stops. The AFO embodiment of the present invention maintains this important benefit, while at the same time supplementing the ankle joint torque to allow for a more powerful push-off.

There is one important mechanical factor to understand before a discussion of the abilities of the present invention can begin: cantilevered springs act exactly like coil springs do. The amount of energy that is stored within them, in the form of potential energy, is directly proportional to the amount of vertical displacement of the free end. An appropriate analogy would be a diving board. As the weight (force) of the diver is applied to the end of the board, it bends and responds with the same force. This means that cantilevered beams are energy conserving. That is, ideally, the amount of the force that was required to displace the beam will be returned when the beam returns to its original shape. Another key mechanical fact to understand is that steel cables can transmit mechanical work only in tension.

A description of the mechanical action of the brace begins with an understanding of the neutral position, which occurs during swing phase. With the foot away from the ground, as in swing phase, the cables 82 & 92 that are connected to either side of the upper ankle joint piece 102 are both in tension. This provides a co-contractile force that maintains the ankle joint in a 90-degree (shank-foot relation) position. This tension is achieved by pre-loading both cantilever springs 40 & 60 to half of the overall displacement positions. Because of the implementation of the pivots 84 & 94, the restorative force that exists in the form of tension in the cables 82 & 92 only acts when the spring returns to its flat position. With both springs 40 & 60 pre-loaded, the restorative force that exists within the springs 40 & 60 is transferred through the pivots 84 & 94 and cables 82 & 92 to the ankle joint 100. The springs 40 & 60 are sized such that the effective spring constant of each is equal and, hence, the restorative forces are equal. By pre-loading both springs 40 & 60, the proper alignment of the ankle joint 100 is ensured, while at the same time allowing for plantarflexion and dorsiflexion to occur. This is because the neutral position is set to be at the midway point of the vertical displacement of the springs. The springs have the ability to return to a flat position or to further displace to a fully flexed position. A mechanical stop prevents the springs 40 & 60 from flexing beyond the yield strength of the material.

Foot strike follows swing phase and begins the process of energy absorption and return. At foot strike the force of the subject striking the ground is applied to the spring 40 in the energy storage component 16. This causes the spring 40 to deflect to its fully-deflected position. As the spring 40 is deflected, the ratcheting teeth 88 of the rear pivots 84 become enmeshed with their respective pawls 86. This process prevents the pivot 84, and thereby the spring 40, from returning to its previous position immediately. This is the point at which the energy of foot strike has been captured and is awaiting release at a later point within the gait cycle. With the spring 40 fully deflected, the pre-loaded tension in trigger component 16 cantilever spring 60 that existed in the neutral position is released because the co-contractile force that was provided by the energy storage component spring 40 has been removed. The effect of this is to provide a dorsiflexion torque at the ankle joint 100 and thereby assist the subject through the second rocker. That is, the foot acts as a pivot over which the body center of mass is assisted by the force in the trigger component spring 60.

As second rocker commences and the body center of mass moves forward, the ground reaction force begins to move forward onto the trigger component spring 60. This causes this spring 60 to deflect. When enough weight has been transferred to the spring 60 to fully deflected it, the spring 60 will depress a small rocker arm that is attached to a cable that is in turn attached to the pawl 86. This releases the pawl 86 from the ratchet teeth 88, which allows the energy storage component 16 spring 40 to return to its natural, non-deflected position. This causes the potential energy that had been stored in the deflected spring 40 to be released and transferred through the cables 82 to the ankle joint 100. The effect of this is to create a plantarflexion torque at the ankle joint, thereby supplementing the push-off power of the ankle joint. Because the trigger component spring 60 is fully deflected by the subject's body weight at push-off, there is enough available slack in the system to allow for full ankle range of motion assist. In addition, the supplemental ankle torque is able to assist the forward motion of the subject because it acts through the lever of the foot at toe-off. It is at this point that the energy that was absorbed at foot strike is released to improve the walking power of the subject.

To complete the gait cycle, the subject lifts the foot from the ground in toe-off. This allows the fully deflected trigger component spring 60 to return to a flat position. However, the energy storage component spring 40 is already in a flat position. Instead, what occurs is that both springs 40 & 60 return to the neutral position (partially deflected) in preparation for the next step.

This AFO design and method are comprised of several key components that are adaptive to different body characteristics for different users. For example, an important body characteristic is weight. In order to maintain the appropriate range of motion (6-8 degrees of plantar/dorsi flexion) and keep the goal of 0.4 Nm/kg of supplemental ankle torque it is imperative that the AFO maintain the same vertical displacement for each of the cantilevered springs 40 & 60. To do this, while still maintaining the ability of the AFO to be effective over a range of body mass, fulcrums 48 & 62 are located beneath the cantilever springs 40 & 60. This allows for an adjustment of the fulcrum point of the springs. With a heavier individual the fulcrums 48 & 62 are moved further from the fixed ends 42 & 58 of the springs 40 & 60. This creates a situation in which the amount of force required to displace the beam is increased allowing for a heavier individual to displace the beam an equal amount as would a lighter individual with the fulcrums 48 & 62 closer to the fixed ends of the springs 42 & 58.

The mechanisms described above are designed to be molded into a custom AFO and incorporated into the sole of a custom shoe. In one exemplary embodiment, the overall height of the leaf spring arrangement is 2.75 cm and the maximum deflection of the leaf spring will be 1.2 cm. This corresponds to 16 degrees of angular motion at the ankle joint 100. The plastic of the brace is the standard polypropylene that is the norm on existing AFOs. The mechanism will add an estimated mass of 0.5 kg to the weight of an AFO.

The invention may be embodied in other specific forms without departing from the spirit or essential characteristics thereof. The foregoing embodiments are therefore to be considered in all respects illustrative rather than limiting of the invention described herein. The scope of the invention is thus indicated by the appended claims rather than by the foregoing description, and al changes which come within the meaning and range of equivalency of the claims are therefore intended to be embraced herein.

One skilled in the art can appreciate that many other embodiments of detection system and method, and other details of construction constitute non-inventive variations of the novel and insightful conceptual means, system and technique which underlie the present invention.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein. We claim:

1. An orthotic device for use on a subject during one or more movement cycles comprising:
an energy storage component which stores mechanical energy generated by the forces between the subject and a supporting surface during a portion of the movement cycle, and
a trigger component which prevents the release of the stored mechanical energy until a specific point in the movement cycle of an extremity of the subject.

2. The device of claim 1, wherein said movement cycle comprises a gait cycle.

3. The device of claim 2, wherein said gait cycle comprises at least one of walking cycle, jogging cycle or running r cycle, or any combination thereof.

4. The device of claim 1, wherein said release of the stored mechanical energy provides a force which aids in the locomotion of the patient.

5. The device of claim 1, wherein said device is an ankle-foot orthosis (AFO).

6. The device of claim 5, wherein said AFO is expanded up the leg.

7. The device of claim 5, wherein said AFO is used in a prosthetic foot and/or leg.

8. The device of claim 1, wherein said device is an arm or hand related orthosis.

9. The device of claim 1, wherein said released mechanical energy assists in plantar flexion.

10. The device of claim 1, wherein said release of stored mechanical energy occurs when the body weight of the subject is over the forefoot.

11. The device of claim 1, wherein said release of stored mechanical energy occurs when the body weight of the subject is at least partially over the forefoot.

12. The device of claim 1, wherein said energy storage component is comprised of at least one spring.

13. The device of claim 12, wherein said at least one energy storage component spring comprises a cantilever spring.

14. The device of claim 1, wherein said trigger component is actuated by a spring reaching a predetermined deflection.

15. The device of claim 14, wherein said spring from which said trigger component is actuated by a cantilever spring.

16. The device of claim 1, wherein said energy storage component has a spring constant, maximum displacement, or initial displacement that is adjustable.

17. The device of claim 16, wherein said energy storage component comprises:
a cantilever spring, and
a fulcrum supporting said cantilever spring.

18. The device of claim 17, wherein said fulcrum has a position that is adjustable to change the spring constant.

19. The device of claim 1, wherein said release of stored mechanical energy is time adjustable.

20. The device of claim 19, wherein said trigger component comprises:
a cantilever spring, and
a fulcrum supporting said cantilever spring.

21. The device of claim 20, wherein said fulcrum has a position that is adjustable to change the spring constant

22. A method of using a orthotic device disposed on a subject, said method comprising:
storing mechanical energy generated by the forces between the subject and a supporting surface during a portion of a movement cycle, and
preventing the release of the stored mechanical energy until a specific point in the movement cycle.

23. The method of claim 22, wherein said storage of mechanical energy is provided by an energy storage component.

24. The method of claim 22, wherein said release prevention is provided by a triggering component.
